(19) United States
(12) Patent Application Publication
(10) Pub. No.: US 2025/0288323 A1
(43) Pub. Date: Sep. 18, 2025

(54) CHEST BOLT AND THORACOSTOMY BOLT

(71) Applicant: SPINTHYR TECHNOLOGIES, LLC, Cambridge, MA (US)

(72) Inventor: Michael George DeBusk, Sturbridge, MA (US)

(21) Appl. No.: 18/861,976

(22) PCT Filed: May 2, 2023
(86) PCT No.: PCT/US23/66512

§ 371 Date: Oct. 31, 2024

Related U.S. Application Data
(60) Provisional application No. 63/337,338, filed on May 2, 2022.

(51) Int. Cl. A61B 17/34; A61B 90/00

(52) U.S. Cl. CPC A61B 17/3494; A61B 17/3415; A61B 17/3417; A61B 90/39; A61B 2017/3492; A61B 2090/3966

(57) ABSTRACT

A chest tube device designed for rapid deployment includes a chest tube and a stylet removably positioned in the lumen of the chest tube. The chest tube includes a flange between the proximal and distal ends to limit insertion of the chest tube into the patient's s body during deployment. At least one strap can be coupled to the flange and secured to the patient to inhibit or prohibit relative movement of the chest tube and patient.

### RELATED APPLICATION(S)

This application claims the benefit of priority to U.S. Provisional Application No. 63/337,338, filed May 2, 2022, which is incorporated herein by reference in its entirety.

### TECHNICAL FIELD

The present disclosure relates generally to thoracostomy tubes, and in particular, to tubes for draining excess fluid from the pleural cavity as a response to trauma.

### BACKGROUND

Tube thoracostomy involves inserting a tube (also known as a chest tube) through the chest wall into the pleural cavity to drain air, blood, bile, pus, or other fluid accumulations resulting from disease and/or a severe injury to the chest wall. Placement of the chest tube allows for continuous, large volume drainage until the underlying pathology can be more formally addressed. The chest tube may be connected to a one-way valve or a suction machine to assist with drainage.

The present application relates to a chest tube designed for safe and rapid deployment in any number of emergent situations.

### SUMMARY

According to certain aspects of the present disclosure, a device for performing a tube thoracostomy is disclosed.

In one embodiment, a chest tube device comprises a chest tube having a lumen extending therethrough from a proximal end of the chest tube to a distal end of the chest tube. The chest tube includes a flange between the proximal end and the distal end to limit insertion of the chest tube into a patient's body during deployment. The chest tube device also has a stylet with a handle and a shaft, the shaft having a proximal end connected to the handle and a free opposite end with a beveled tip. The shaft of the stylet is slidably positioned in the lumen of the chest tube with the beveled tip protruding from the distal end of the chest tube to facilitate insertion of the chest tube into the patient's body, wherein the stylet is removable from the chest tube after deployment.

In an alternative embodiment, a chest tube device comprises a chest tube having a proximal end and a distal end, and a lumen extending therethrough, the proximal end having an opening with a diameter larger than a diameter of the lumen (i.e., flared) and the distal end having a tapered tip. The device further comprises a flange having an inner diameter equal to an outer diameter of the chest tube and positioned on the chest tube to limit insertion of the chest tube into a body during deployment of the chest tube device, the flange having at least one cutout for a strap to fix the chest device to the body. A stylet is also included, the stylet having a proximal end and a distal end, the proximal end having a handle for a user to grip during deployment, the distal end having a beveled and sharpened tip, the stylet further having a diameter smaller than a diameter of the lumen of the chest tube, and wherein the stylet is inserted through the chest tube such that the distal end of the stylet protrudes from the distal end of the chest tube, and the handle of the stylet is proximal to the proximal end of the chest tube.

In another embodiment, a method comprises inserting a stylet into a chest tube so that a distal end of the stylet is distal to the distal end of the chest tube and a handle of the stylet is proximal to a proximal end of the chest tube, the chest tube having a circular flange attached to a strap. The stylet is slidably advanced through the chest tube to an insertion site on a patient, upon which the stylet punctures skin of the patient, thereby inserting the chest tube device into the patient's chest cavity. The chest tube device position at the insertion site is secured by wrapping the strap across the patient's torso, and air and/or liquid is drained from the chest cavity through the chest tube device.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments.

FIG. 1A illustrates an exemplary chest tube device in accordance with one or more embodiments.

FIG. 1B illustrates the chest tube portion of the chest tube device of FIG. 1A.

FIG. 1C illustrates the stylet portion of the chest tube device of FIG. 1A.

FIG. 2A illustrates examples of placement of the chest tube through an anterior chest wall.

FIG. 2B shows the inside view of the anterior chest wall corresponding to FIG. 2A.

FIG. 3 illustrates an alternative exemplary chest tube in accordance with one or more embodiments.

FIG. 4 illustrates an alternative exemplary stylet in accordance with one or more embodiments.

FIG. 5 illustrates another alternative exemplary chest tube in accordance with one or more embodiments.

FIG. 6 illustrates another alternative exemplary chest tube device in accordance with one or more embodiments.

FIGS. 7-9 illustrate further alternative exemplary stylets in accordance with one or more embodiments.

FIG. 10 illustrates an alternative exemplary right-angle tube in accordance with one or more embodiments.

FIG. 11 illustrates examples of placement of the right-angle tube through an anterior chest wall in accordance with one or more embodiments.

FIG. 12 shows the inside view of the anterior chest wall corresponding to FIG. 11.

FIG. 13 illustrates an example of placement of the right-angle tube through the cricothyroid membrane for emergency cricothyroidotomy.

FIG. 14A illustrates an alternative exemplary chest tube device in accordance with one or more embodiments.

FIG. 14B illustrates the chest tube portion of the chest tube device of FIG. 14A in accordance with one or more embodiments.

FIG. 14C illustrates the stylet portion of the chest tube device of FIG. 14A in accordance with one or more embodiments.

FIG. 15 illustrates the stylet portion of the chest tube device of FIG. 14A slidably positioned within the lumen of the chest tube portion along its entire length, in accordance with one or more embodiments.

FIG. 16 illustrates an example of how the chest tube device of FIG. 14A may be held in the hand, in accordance with one or more embodiments.

FIG. 17A illustrates examples of placement of the chest tube device through an anterior chest wall, in accordance with one or more embodiments.

FIG. 17B shows the inside view of the anterior chest wall corresponding to shows the inside view of the anterior chest wall corresponding to FIG. 5A.

FIG. 18A illustrates a view of the exemplary distal stylet tip of FIGS. 14A and 14C in accordance with one or more embodiments.

FIG. 18B illustrates a view of the exemplary distal stylet tip of FIGS. 14A and 14C in accordance with one or more embodiments.

FIG. 18C illustrates an example of placement of the stylet of FIGS. 14A and 14C through a chest wall and function of the distal stylet tip in accordance with one or more embodiments.

FIG. 19 illustrates an alternative exemplary chest tube portion of the chest tube device in accordance with one or more embodiments.

FIG. 20 illustrates an alternative exemplary chest tube portion of the chest tube device in accordance with one or more embodiments.

FIG. 21 illustrates an alternative exemplary chest tube portion of the chest tube device in accordance with one or more embodiments.

FIG. 22 illustrates an alternative exemplary chest tube portion of the chest tube device in accordance with one or more embodiments.

FIG. 23 illustrates a further alternative exemplary chest tube in accordance with one or more embodiments.

FIG. 24 illustrates an alternative exemplary chest tube portion of the chest tube device of FIG.14A and 14B in accordance with one or more embodiments.

FIG. 25 illustrates a further alternative exemplary chest tube portion of the chest tube device of FIG. 14A and 14B in accordance with one or more embodiments.

FIG. 26 illustrates the flange portion of the Chest Bolt of FIG. 14A and 14B in accordance with one or more embodiments.

FIG. 27 illustrates an alternative exemplary flange portion in accordance with one or more embodiments.

FIG. 28 illustrates an alternative exemplary flange and chest tube in accordance with one or more embodiments.

FIG. 29 illustrates a further alternative exemplary flange and chest tube in accordance with one or more embodiments.

FIG. 30 illustrates an alternative exemplary right-angle chest tube device in accordance with one or more embodiments.

FIG. 31A illustrates examples of placement of the right-angle tube through a chest wall in accordance with one or more embodiments.

FIG. 31B shows the inside view of the chest wall corresponding to FIG. 31A.

FIG. 32 illustrates placement of the right-angle tube through the anterior neck into the trachea for an emergency airway (i.e., cricothyroidotomy).

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

Fluid draining is a procedure in which a body area or organ is punctured by a needle, or an incision is made to drain fluid accumulated in the body area. The fluid includes liquid, gas, cellular material, or a combination thereof. Fluid draining can be used as a therapeutic measure where some volume of fluid is removed from the body area to lessen discomfort or prevent spread of discomfort.

A process of fluid draining involves making an incision, inserting a device through the incision site, and removing the excess fluid through the device. The device is inserted to a desired depth to sufficiently drain the fluid. The device, referred to as a chest tube, thoracostomy tube, or thoracic catheter, is a flexible tube that can be inserted through the chest wall between the ribs into the pleural space. The pleural space is the space between the parietal and visceral pleura, and is also known as the pleural cavity. A patient may require a chest drainage system as a result of trauma, for example when the negative pressure in the pleural cavity is disrupted, resulting in respiratory distress. A small amount of fluid or air may be absorbed by the body without a chest tube. A large amount of fluid or air cannot be absorbed by the body and generally requires a drainage tube. If incorrectly inserted at a depth too shallow or too deep, excess fluid may not be correctly drained from the site.

The chest tube can be connected to a closed chest drainage system, which allows for air or fluid (e.g. liquid) to be drained, and prevents air or fluid from entering the pleural space. The system is airtight to prevent the inflow of atmospheric pressure. Because the pleural cavity normally has negative pressure, allowing for lung expansion, any tube connected to it must be sealed so that air or liquid cannot enter the space where the tube is inserted.

The location of the chest tube depends on what is being drained from the pleural cavity. If air is in the pleural space, the chest tube is inserted above the second intercostal space at the midclavical line. If there is fluid in the pleural space, the chest tube is inserted at the fourth to fifth intercostal space, at the mid-auxillary line. A chest tube may also be placed directly under the sternum. Once a sufficient volume of fluid is drained, the chest tube is removed from the incision site.

Embodiments of the present disclosure describe a chest tube device for performing fluid draining. The chest tube device can be implemented for insertion between the ribs and draining excessive liquid from the patient's upper chest resulting from trauma. To determine proper insertion depth of the device, and/or prevent excessive insertion, the chest tube has a flange. The flange is located along the body of the chest tube and prevents full insertion of the chest tube into the incision site.

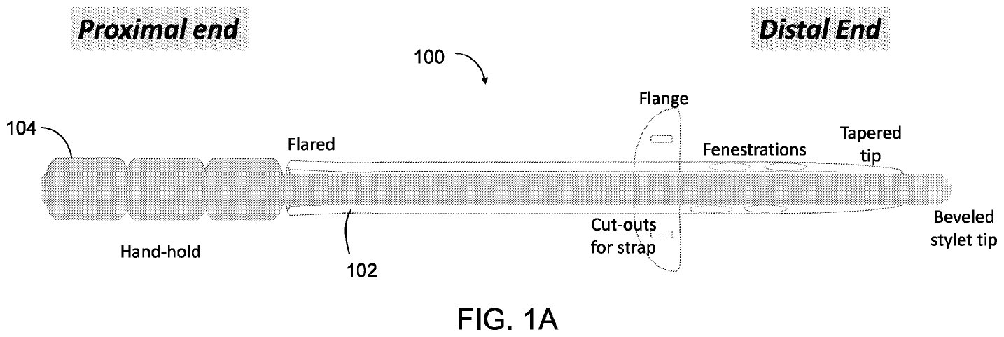

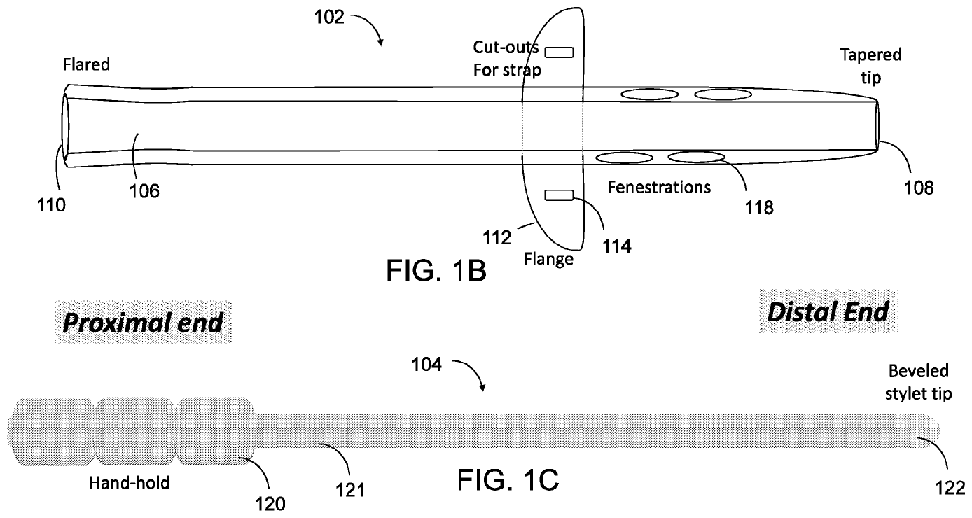

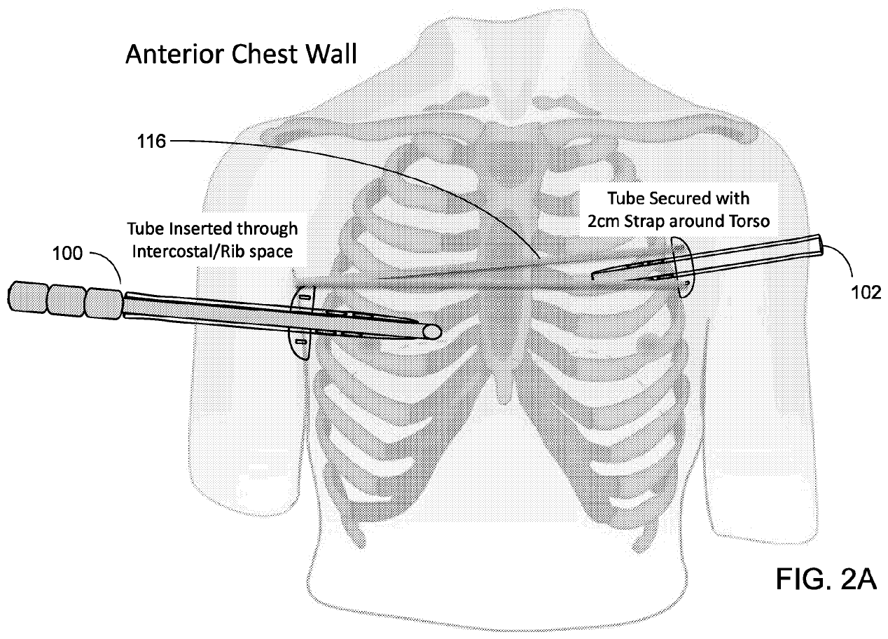

FIGS. 1A-1C illustrate an exemplary chest tube device 100 designed for rapid deployment in accordance with one or more embodiments. The chest tube device 100 includes a chest tube 102 (shown separately in FIG. 1B) and a stylet 104 (shown separately in FIG. 1C). The stylet 104 is removably positioned in a lumen 106 in the chest tube 102.

The chest tube 102 may be used by an operator, such as a physician or another medical personnel, operating the chest tube device 100 to remove fluid from an aspiration or an incision site on a patient's body. The chest tube 102 can have a plastic or steel composition. In some embodiments, the chest tube 102 comprises a flexible material allowing for bending of the chest tube to better accommodate body parts and tube-articulation to access hard to reach areas subject to fluid buildup. Chest tube 102 can range in size, e.g. from 6 French to 40 French, and is fenestrated along sides of the insertion end. In some embodiments, chest tube 102 has a radio-opaque stripe located along one edge of the chest tube 102 to help identify the device on radiographic imaging (e.g., chest X-Rays). In another embodiment, the stripe may also be phosphorescent for use in low-light conditions. In some embodiments, chest tube 102 has a series of measurements on the exterior of the tube to display to the user the depth the tube has been inserted into the patient.

The chest tube 102 includes a proximal end and a distal end. The distal end includes a slightly tapered tip 108 to reduce resistance when inserting the chest tube device 100 through tissue. In some embodiments, the tip 108 has an opening with a diameter of 2 cm, for example,. In some embodiments, the tapering of the tip 108 does not affect the opening diameter of the chest tube 102 and stays constant through the distal end of the device to allow for unrestricted movement through the distal end of chest tube 102. In other words, only the outer diameter of the tube 102 is reduced such that the thickness of the tube wall is reduced at the distal end.

The proximal end of the tube 102 can include a slightly flared opening 110 to allow easier insertion of an adapter for a suction canister once the tube 102 has been deployed (and stylet 104 removed). Suction is generally used to drain liquid through the chest tube 102, but alternative methods such as pressure differentials, gravity, and intubation may be used to drain liquids from the chest cavity through the chest tube as well. If suction is used to drain liquid through the chest tube 102, the adapter for the suction canister can be placed over the flared opening 110. In some embodiments, the diameter of the opening 110 can measure 2.3 cm, increasing from a 2 cm opening at the opposite end, tapered tip 108. In some embodiments, the flared opening 110 can further include other connection mechanisms for a suction canister or other attachments, including a Luer lock, threading, a snap fit connection, or other suitable means.

A flange 112 is included on the exterior of the chest tube 102. The flange 112 can be integrally formed with the chest tube, or provided as a discrete component that is coupled to the exterior surface (e.g. via mechanical or adhesive bonding). In some embodiments, the flange 112 is circular (e.g. circumscribes the tube 102) and placed approximately 8 to 10 cm from the distal tip 108. In some embodiments, the flange 112 has an exterior diameter of 7 cm and a thickness of 1.5 cm. In some embodiments, the flange 112 is a oval shape with a circular cutout with a diameter corresponding to the exterior diameter of the chest tube 102. The flange 112 acts as an anchor against the patient's skin at the incision site to prevent further travel of the chest tube 102 through the chest wall during deployment. In some embodiments, the circular flange 112 is permanently fixed to the exterior of chest tube 102. For example, the permanently fixed flange 112 may provide a stable and steady anchor for the chest tube 102.

In some embodiments, the flange 112 can be adjusted along the exterior of the chest tube 102 so the user is able to effectively drain liquid from different depths within the pleural cavity (and/or be adjusted in size to accommodate different patient's anatomy). In these embodiments, this adjustable flange 112 also allows the chest tube 102 to be used for a wider variety of sizes of patients. For example, the flange 112 can be adjusted to sit closer to the distal end of the chest tube 102 for smaller patients or children. Likewise, the flange 112 can be adjusted to sit closer to the proximal end of the chest tube 102 for larger patients, or patients with liquid deeper within the pleural space. In some embodiments, the chest tube 102 has a series of surface features (e.g. indents and/or protrusions) along the exterior body. The adjustable flange 112 has a corresponding surface feature (e.g., slots and/or protrusions) on its circular interior body, such that the indent of the chest tube 102 mates with its corresponding slot on the adjustable flange 112. When the flange 112 is in place along the chest tube 102, haptic feedback is provided to the user to indicate that the flange 112 has been properly adjusted. Alternatively, the user is able hear the flange 112 click into place, or a visual indication may be present.

The flange 112 has cutouts 114 for attachment of a strap 116 to be fit around the torso once the chest tube 102 has been deployed. The cutouts 114 can be situated opposite one another on the flange 112. In some embodiments, two cutouts 114 are located equidistantly from the tube 102 exterior, thereby providing a uniform load path in each strap 116 and evenly distributing the forces exhibited on the tube 102. The cutouts 114 can vary in size/shape/dimension to accommodate straps of different thicknesses, widths, or materials. In some embodiments, the cutouts 114 are a standard rectangular size. In alternative embodiments, the cutouts 114 can be circular, oval, or square in shape. Regardless of shape or dimension, each of the cutouts 114 are centered on their respective flange 112. The straps can be removably coupled to the flange 112, or permanently attached thereto. Also, the strap(s) 116 can be adjustable in length.

The strap(s) 116 can engage the flange 112 such that the entire distal surface of the flange abuts the patient. This provides a uniform pressure distribution across the surface of the flange 112. In some embodiments the straps can be tightened (e.g., reduced in length) to further deploy the tube 102 within the patient.

In some embodiments, the strap 116 is a 2 cm cotton strap. One end of the strap 116 is in a fixed position in one of the cutouts 114 in the flange 112. The opposite free end of the strap 116 can be wrapped around the torso and attached to the other cutout 114 to fix the chest tube 102 in place on the torso. The strap 116 secures the chest tube 102 to the patient and keeps the chest tube 102 from excessive movement or removal from its fixed position at the incision site. The strap 116 can be wrapped around any area of the torso, and is of a suitable length to allow for placement of the chest tube device 100 at any position on the patient's chest. In some embodiments, the strap 116 comprises a plastic, a rubber, a gauze, or any other suitable material to hold the chest tube 102 in place. The straps 116 can be a single contiguous length of material that is looped around the body and secured to the flange; alternatively, a plurality of straps 116 of discrete material can be employed to secure the flange 112.

In some embodiments, the chest tube 102 includes one or more fenestrations 118 between the flange 112 and the tapered tip 108 for egress of fluids. Each of the one or more fenestrations 118 can be situated around the body of the chest tube 102 and made of a variety of shapes/sizes (e.g. circular, oval, elliptical, linear, etc.). In some embodiments, the fenestrations 118 are parallel to one another. In some embodiments, the fenestrations are randomly placed around the body of the chest tube 102, but always remaining in the distal portion of the body of the chest tube 102 between the flange 112 and the tapered tip 108. In some embodiments, the fenestrations 118 have a diameter of 1.5 cm. In some embodiments the fenestrations are of uniform size/shape; in other embodiments the fenestrations have a varied size/shape (e.g. smaller openings closer to the distal end of the tube 102).

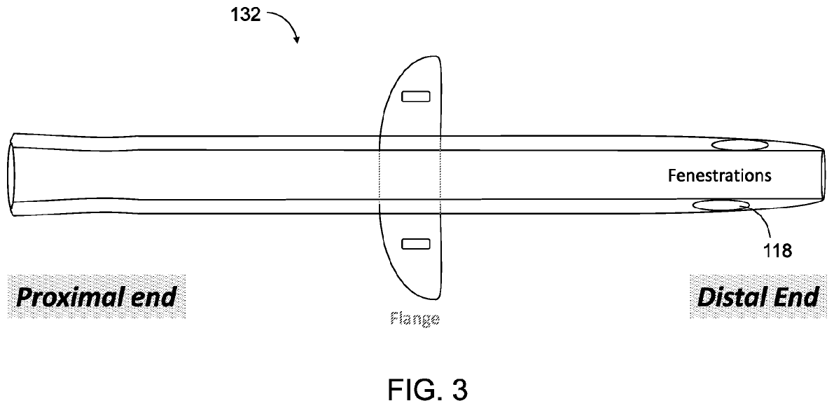

As shown in the chest tube embodiment 132 of FIG. 3, the fenestrations 118 can comprise a hole through the body of the chest tube 102 about 6 cm distal of the flange 112 towards the tapered tip 108 of the chest tube 102.

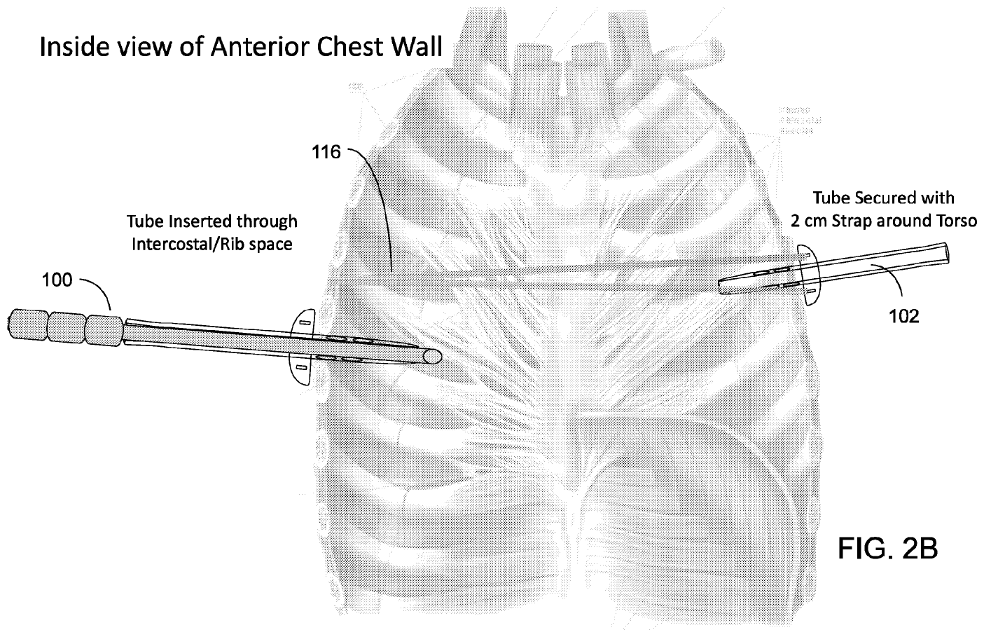

The chest tube 102 includes a lumen 106 extending along its entire length, in which the shaft of the stylet 104 is slidably positioned as shown in FIGS. 1A and 2. In some embodiments, the lumen 106 has a constant diameter throughout the body of the chest tube 102. In some embodiments, the diameter of lumen 106 is wider at the proximal end of chest tube 102 to allow for connection to a suction device, for example. Liquid can move into the lumen 106 through the distal end of the chest tube 102, and/or through the fenestrations 118.

The chest tube 102 can comprise a soft material, e.g., a soft silastic plastic (a flexible, inert silicone elastomer). Alternatively, the chest tube 102 can comprise PVC, silicone, metal, steel alloy, other flexible plastics, vinyl materials, or any suitable combination thereof. In some embodiments, the chest tube 102 is reusable and comprises a material that can be autoclaved or sanitized by a user.

The stylet 104 can comprise a one-piece, solid structure made of a plastic material, e.g., high-density polyethylene (HDPE). The stylet 104 is dimensioned to be slidably deployed through the lumen 106 of the chest tube 102. The stylet 104 includes a handle 120 at the proximal end. Stylet 104 can be removably connected to handle 120. The stylet 104 has shaft 121 connected to the handle 120 with a beveled distal tip 122 to facilitate insertion through the chest wall. When the stylet 104 is positioned in the chest tube 102 and deployed by the user sliding the stylet 104 through the lumen 106, the distal tip 122 of the stylet 104 protrudes from the distal end 108 of the chest tube 102, such that the beveled tip 122 functions like a knife-edge to cut through skin and separate tissue. The sharpened edge of the tip 126 functions as a knife to cut through skin and fibrous tissue decreasing the effort of inserting the chest tube device 100.

The chest tube device 100 (with the stylet 104 positioned in the chest tube 102) is designed to be rapidly deployed together as a single unit for insertion through the chest wall. The chest tube device 100 obviates need for any other additional instruments for deployment. Thus, the drainage tube 102 is inserted to the desired location/depth simultaneously with the piercing step, thereby reducing complexity, discomfort, and risk of contamination. Chest tube device 100 can be connected to a suctioning system or other operating equipment to effectively drain the pleural cavity of the patient.

The chest tube device 100 is easy to use and simple in design. The chest tube device 100 is easily transported and stored, and can be reusable. In some embodiments, each element of the chest tube device 100 can be disconnected and sterilized before reusing. Trainees can learn and rapidly deploy the device 100 with little training, and are able to quickly assemble the device 100 during high-pressure situations due to the simple, inclusive design.

The handle 120 of the stylet 104 has a raised, undulating profile so that it can be easily and securely grasped by a user's hand. The handle 120 allows for easier grasping even when wet. It provides a more comfortable control of chest tube device 100. The handle 120 can comprise a foam, plastic, or rubber material. The texture of the handle 100 provides for an easy to grip surface to allow for easy use of the stylet 104 and chest tube device 100. In some embodiments, handle 120 is configured to be hammered into place as shown in FIG. 8, or can be attached to a security eyelet 146 as shown in FIG. 9.

The stylet 104 can also be used separately from the chest tube 102 as a utility knife, including as a stand-alone defensive tool due to its beveled distal tip 122. In addition, when field-cleaned, the stylet 104 can be re-used with other chest tubes 102. The stylets disclosed herein are thus multi-functional and can have a variety of uses in addition to their use in deploying chest tubes. The stylets disclosed herein can be packaged together with chest tube devices or separately.

As shown in FIG. 2A, the chest tube device 100 may be deployed in a variety of positions around the pleural cavity. As shown on the left hand portion of FIG. 2A, the device 100 can be deployed to insert the chest tube 102 through the intercostal rib space without use of the strap 116 to maintain the placement of the device 100. Alternatively, and as shown on the right hand portion of FIG. 2A, the strap 116 secures the chest tube 102 at the insertion site by running around the torso of the patient. FIG. 2B shows the inside view of the anterior chest wall corresponding to FIG. 2A.

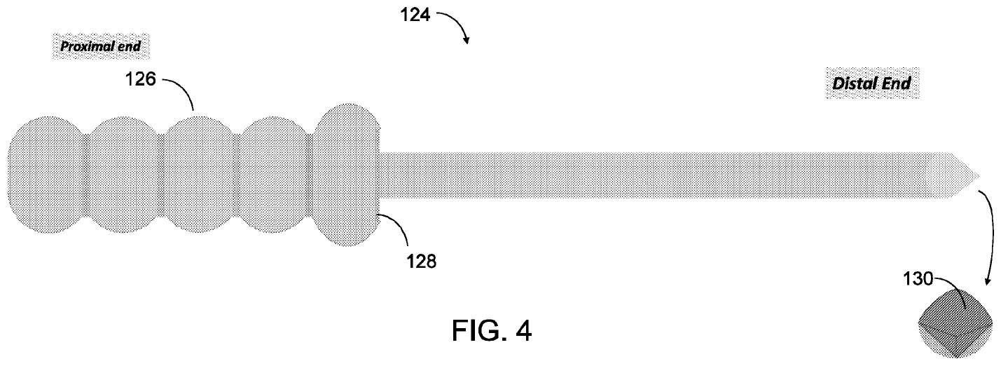

FIG. 4 illustrates an alternate stylet 124 in accordance with one or more embodiments. The stylet 124 includes a handle 126 that is knurled (i.e., with finger grooves) for easier gripping. The distal knurl is larger than other knurls similar to the hilt of a sword. This provides a tactile response and resting surface for the thumb and index finger during insertion. This also provides a tactile indication of the end of the handle 126 in cases where the user isn't able to see clearly or needs to quickly insert the stylet 124.

The distal end of the handle 126 is flat and curved slightly inwards at the edges 128 to engage and accommodate the proximal end of the chest tube 102. This configuration provides a flat surface against the chest tube 102 for insertion and ease of use during deployment, when the user pushes the stylet 124 through the chest tube 102 to drain liquid from the pleural space. The contoured depression 128 in the handle 120 of the stylet 104 and contoured depression 130 in the proximal end of the chest tube 102 are ergonomically fashioned to fit easily and securely within the user's hand when grasped (shown by way of example in FIG. 3). The contoured depressions in the stylet handle 128 and proximal end 130 of the chest tube 102 together allow for easier grasping even when soiled. These exemplary embodiments provide for a greater and more comfortable control of the chest tube device 100 coupled through with an instinctive haptic feedback, improved security from slippage and intuitive deployment of the chest tube device 100.

In some embodiments, the stylet distal tip 130 is cut in a triangular shape below the bevel. This provides a point to easily cut through the skin during deployment of the chest tube device 100. While not shown, it is contemplated that the stylet distal tip 130 can comprise other shapes or points to better cut through the skin when slidably deployed through the chest tube 102.

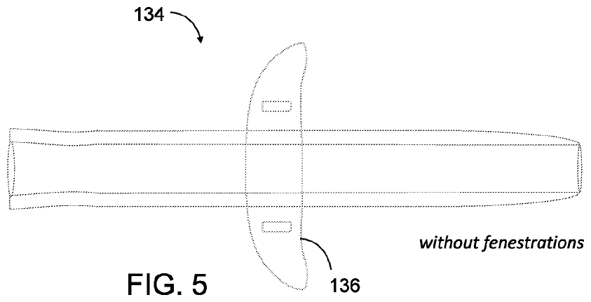

In the FIG. 5 chest tube embodiment 134, there are no fenestrations. In this embodiment, liquid is only drained through the lumen 106 of the chest tube 134. In addition, the surface of the flange 136 is concave (i.e., not flat with respect to the distal portion of the chest tube) with edges curved slightly inward toward distal end 108. This surface shape confers added rigidity to the edges to increase resistance to insertion as feedback during placement. For example, a user is able to place the chest tube 134 more securely against the patient's skin at the insertion site, as the edges of the flange 136 serve to stabilize the chest tube 136. This surface shape also resists eversion of flange 136 and distal migration of chest tube 134.

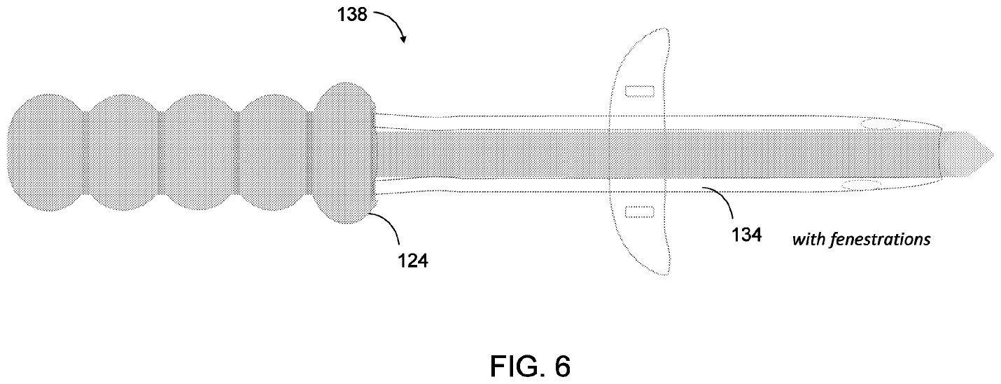

FIG. 6 illustrates a chest tube device 138 comprising the tube 134 of FIG. 5 and the handle 124 of FIG. 4. Here, the chest tube device 138 has a series of fenestrations 118 between the distal end of the flange 112 and distal end of the chest tube 134, allowing for movement of liquid into the chest tube 134.

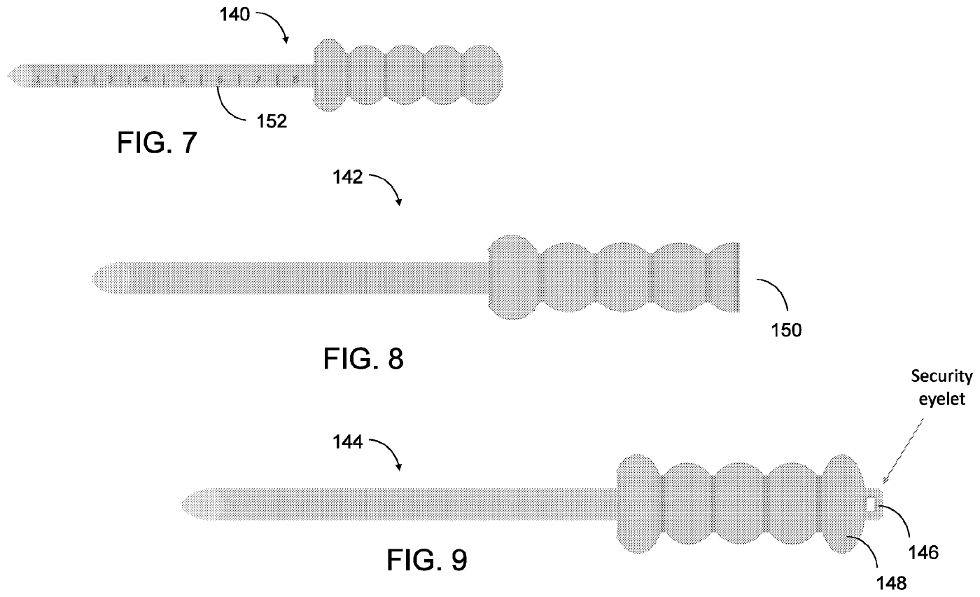

FIGS. 7-9 illustrate further alternative exemplary stylets 140, 142, 144 in accordance with one or more embodiments. The stylets 140, 142, 144 comprise “utility” or “multi-tool” devices having multiple uses.

In the embodiment shown in FIG. 7, a luminescent ruler 152 (e.g., in cm) is embossed on stylet shaft. The markings are phosphorescent for use in low-light conditions. Gradations measured starting from cutting tip. The ruler can be used to indicate how deep into the pleural cavity the stylet 140 has been inserted. Additionally, the chest tube 102 used with stylet can be opaque or clear to allow for the user to read measurements from the ruler while deploying the stylet 140.

In the embodiment shown in FIG. 8, stylet 142 has a butt end 150 at the proximal end of the stylet 142. The butt end 150 is flat so that it can be more easily hammered for use as a stake or pounding object. This is intended to be used when stylet 142 is not easily insertable into the patient's pleural cavity. Alternatively, the stylet 142 may have uses for which it is necessary to use additional force by hammering to penetrate a surface.

In the embodiment shown in FIG. 9, stylet 144 has a proximal (i.e., “butt”) end of handle including a built-in security eyelet 146 for attaching a strap or cord. This allows fastening the stylet 144 to a person or gear to prevent loss of the stylet 144 or to easily keep track for quick use. The butt end knurl 148 is slightly larger than the rest of the handle to provide tactile feedback for users. In addition, this feature is similar to a sword handle providing an improved grip for quick use. It is envisioned that the stylet 144 is attached to other components of chest tube device 100 for ease of use during stressful situations requiring quick actions.

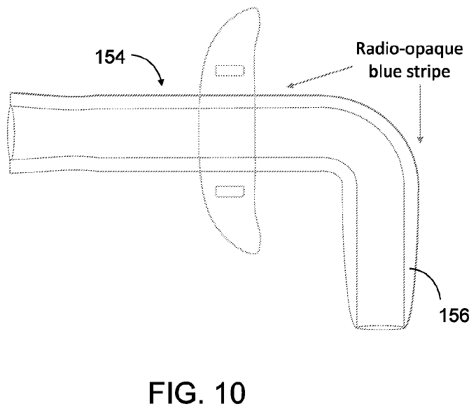

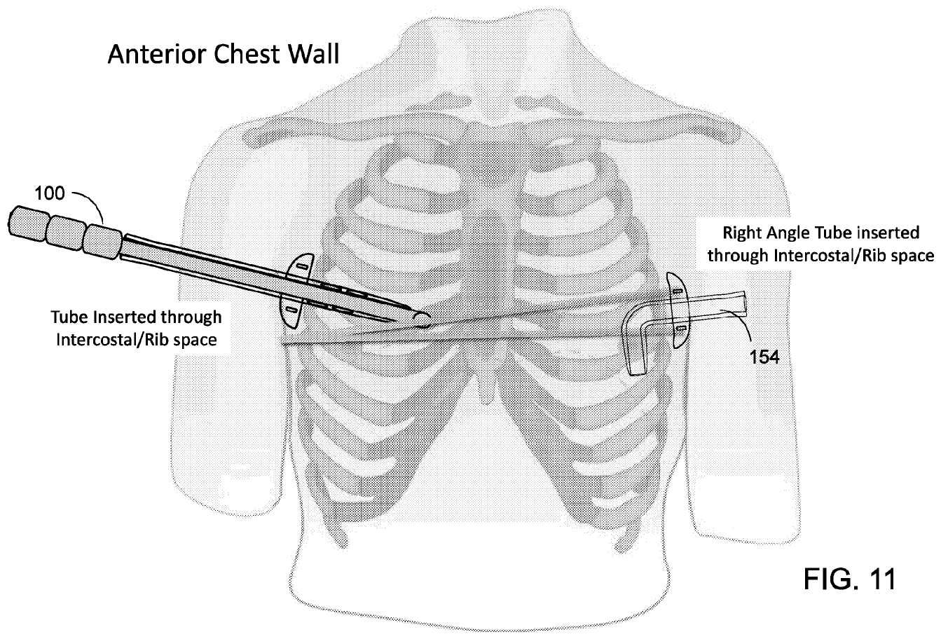

FIG. 10 illustrates an alternative exemplary right-angle chest tube 154 in accordance with one or more embodiments. The tube 154 includes a distal end curved 90′ to allow directionality, for example to be used when liquid within the pleural cavity is not easily accessed by a “straight” chest tube 102 due to ribs or other anatomical structures. Additionally, the chest tube 154 has a flange 112 proximal to the right angle bend in the chest tube 154. In some embodiments, it is conceivable that the flange 112 is distal of the right angle bend in the chest tube. While the embodiment in FIG. 10 does not comprise any fenestration along the distal end of the chest tube 154, alternative embodiments may include fenestrations 118 as shown in previous figures. In some embodiments, the chest tube 154 comprises a bend of less, or more, than 90′. The (straight) stylet 104 can be employed, without a tube 154, to pierce the patient and initiate a pathway for the tube 154 insertion; thereafter the tube 154 can be partially inserted, e.g. up to the bend location, and articulated by the physician to change orientation, and further insertion angle of the tube within the patient such that the tube results in the positioning shown in FIG. 11. Additionally or alternatively, in some embodiments the tube 154 can be originally configured as a straight tube, but upon insertion within the patient, the tube “converts' or articulates to the bent configuration 154, as shown in FIG. 10. For example, the tube can be formed from shape-memory material such that when inserted into the body, the tube transitions from a martensitic (straight) configuration to an austenitic (bent) configuration.

FIG. 11 illustrates exemplary placements of the right-angle tube 154 through an anterior chest wall in accordance with one or more embodiments. Similar to the embodiments as shown in FIGS. 2A and 2B, the exemplary chest tube device 100 and chest tube 154 are inserted through the intercostal rib space to drain liquid within the pleural cavity. Chest tube 154 is secured by strap 116 placed around the patient's torso to ensure stability of the chest tube 154 during deployment.

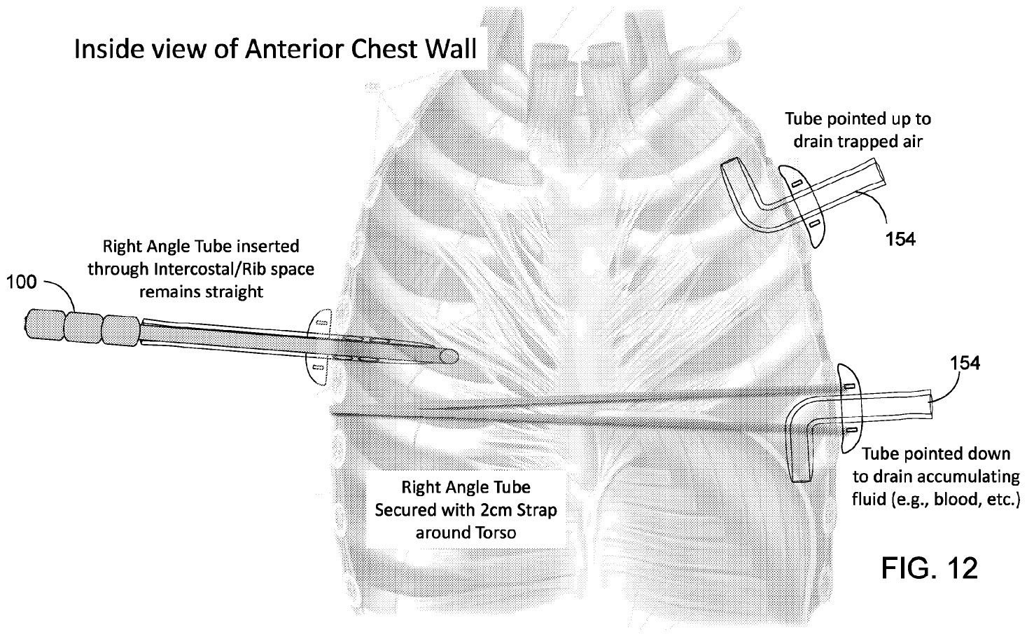

As shown in FIG. 12, the tube 154 can be positioned cephalad (i.e., towards the head or “up”), which allows for insertion pointing towards trapped air within lung space. The tube 154 can also be positioned caudally (i.e., towards the feet or “down”), which allows pointing towards fluid towards the back of the patient and base also shown in FIG. 12. Depending on the location of fluid within the pleural cavity, it may be necessary to change the orientation of the chest tube to better drain accumulated fluids. In some embodiments, the tube 154 is bent prior to insertion into the pleural cavity. In alternative embodiments, the tube 154 bends in response to anatomical structures within the pleural cavity. In either embodiment, fluid drainage is not interrupted by the bend in the chest tube 154.

As further shown in FIG. 12, some embodiments of the right angle chest tube 154 can remain straight upon insertion. In these situations, the chest tube 154 comprises a stiffer material that does not bend upon meeting anatomical structures.

A radio-opaque blue strip 156 along “back” or “spine” of tube identifies tube curvature. This strip 156 can include a ruler or other measurement system to indicate to the user the insertion depth of the chest tube 154.

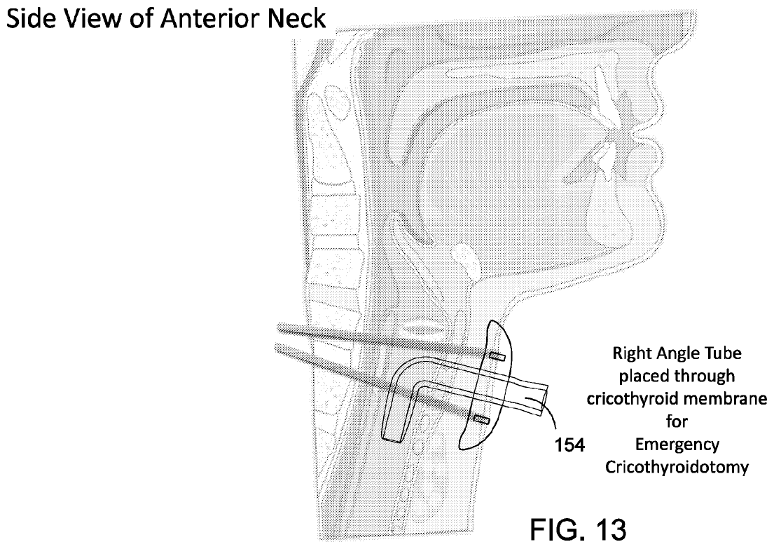

As shown in FIG. 13, the tube 154 can also be used for rapid field tracheostomy (i.e., breathing tube inserted through the anterior neck into the trachea). The tube 154 thereby provides an “all in one” field system of management when combined with the HDPE stylet for airway and breathing issues in the field. A right-angle tube 154 can be used for airways, while a chest tube 102 can be used for breathing. These are two of the most critical aspects of tactical casualty care, in addition to massive hemorrhage/exsanguination.

The right-angle tube 154 can comprise a soft material, e.g., a soft silastic plastic (a flexible, inert silicone elastomer) that has been molded into shape. Alternatively, the tube 154 can comprise a suitable plastic, rubber, or metal as needed. The material of the tube 154 can be sterilized or autoclaved to allow for reuse, or can be intended as a one-time use.

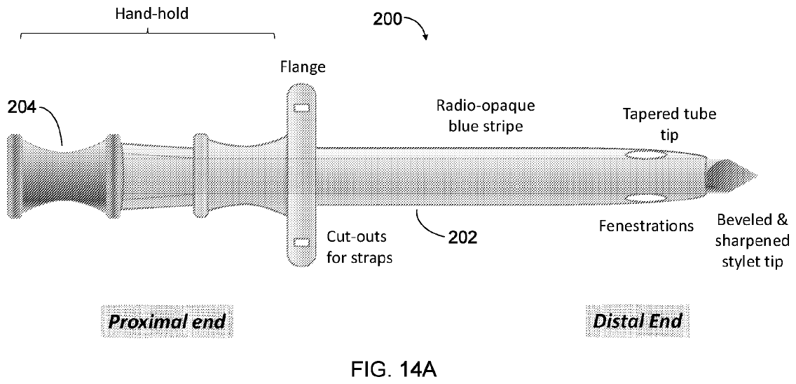

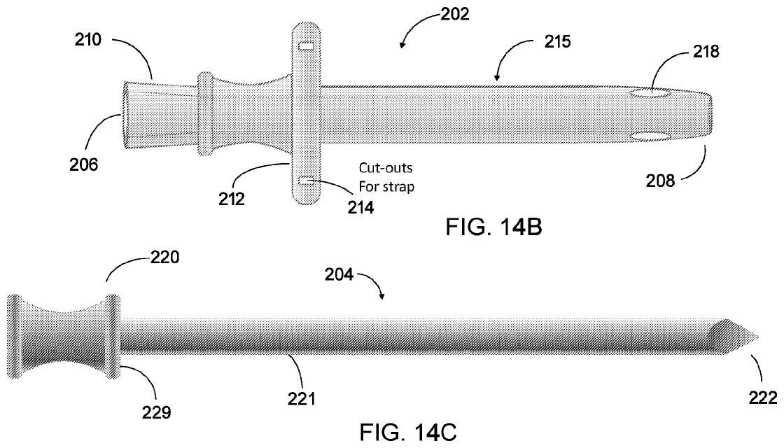

Alternative embodiments of the chest device are shown in FIGS. 14-32.

In FIGS. 14A-C, an exemplary chest tube device 200 designed for rapid deployment is illustrated. The chest tube device 200 includes a chest tube 202 (shown separately in FIG. 14B) and a stylet 204 (shown separately in FIG. 14C). The stylet 204 is removably positioned in a lumen 206 in the chest tube 202.

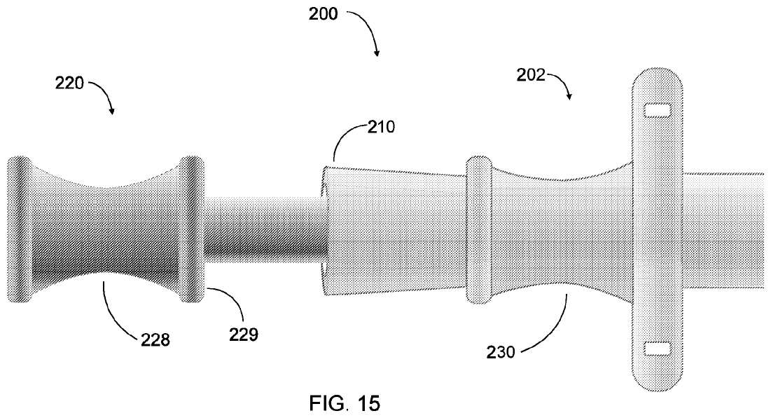

FIG. 15 illustrates an expanded view of the stylet 204 and handle 220.

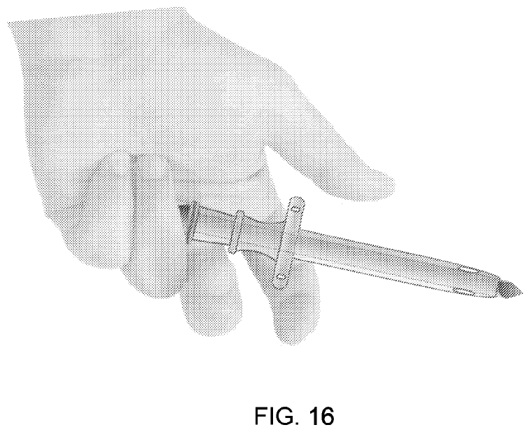

The stylet 204 comprises stylet shaft 221 and handle 220, as well as a beveled and sharpened distal tip 222. The stylet handle 220 can also include a recessed groove or channel 229 in the handle. The contoured depression 228 in the handle 220 of the stylet 204 and contoured depression 230 in the proximal end of the chest tube 202 are ergonomically fashioned to fit easily and securely within the user's hand when grasped (shown by way of example in FIG. 16). A flared portion/end 210 of the chest tube can be positioned between the concave depression 220 and 230, as shown in FIG. 14A. In other words, in the exemplary embodiment shown in FIG. 14, the user grasps both a portion of the stylet 204, and a portion of the chest tube 202 when inserting the assembly into the patient. The contoured depressions in the stylet handle 128 and proximal end 130 of the chest tube 102 together allow for easier grasping even when soiled. These exemplary embodiments provide for a greater and more comfortable control of the chest tube device 200 coupled through with an instinctive tactile response, improved security from slippage and intuitive deployment of the chest tube device 200.

The distal end includes a slightly tapered tip 208 to reduce resistance when inserting the chest tube device 200 through tissue. The proximal end of the tube 212 can include a slightly flared opening 210 to allow easier insertion of an adapter for a suction canister once the tube 202 has been deployed (and stylet 204 removed). The chest tube 202 also includes a radio-opaque stripe 215 along the exterior for identification purposes.

A flange 212 is included on the exterior of the chest tube 202. The flange 212 has cutouts 214 for attachment of a strap 216 to be fit around the torso once the chest tube 202 has been deployed. In some embodiments, the chest tube 202 includes one or more fenestrations 218 between the flange 212 and the tapered tip 208 for egress of fluids.

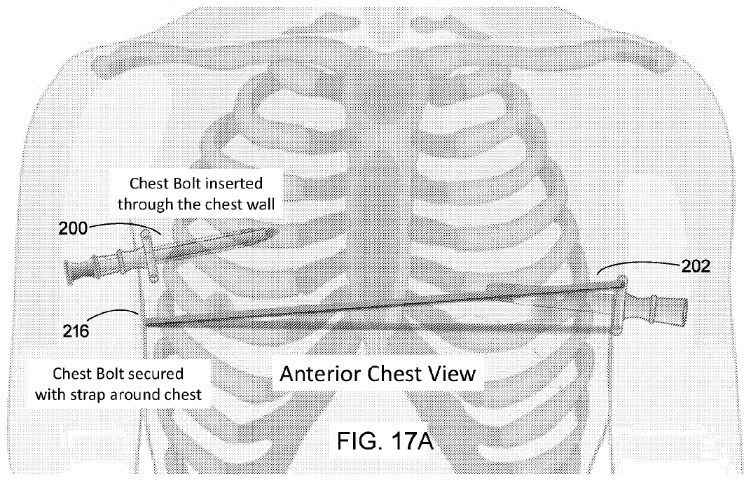

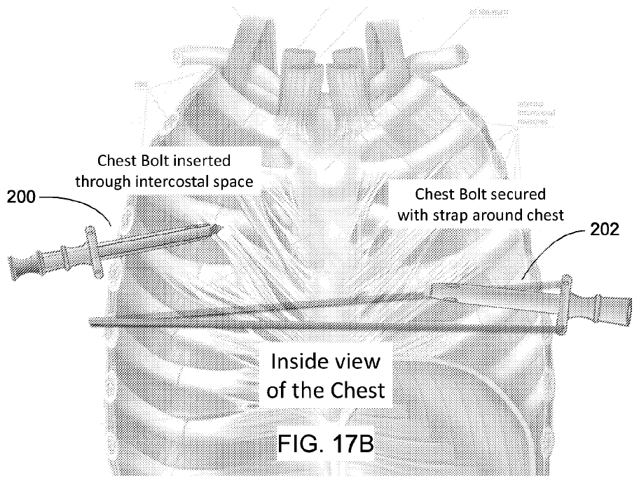

As shown in FIGS. 17A and B, the chest tube device 200 may be deployed, individually or in combination with a plurality of chest tubes, in a variety of positions around the chest wall. FIG. 17A shows an anterior view of the chest with the chest tube device 200 inserted. The stylet portion 204 is withdrawn by sliding it out of the lumen 206 of the chest tube portion 202. The chest tube portion 202 is then secured, for example, using a strap 216 fastened to the cutouts 214 in the chest tube flange 212. FIG. 17B shows the inside view of the chest and deployed chest tube device 200.

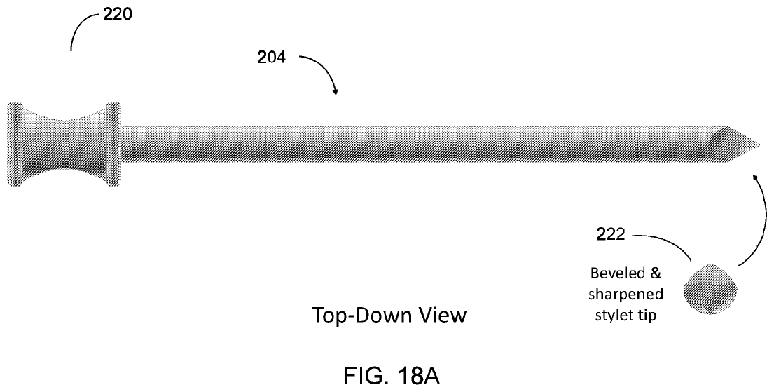

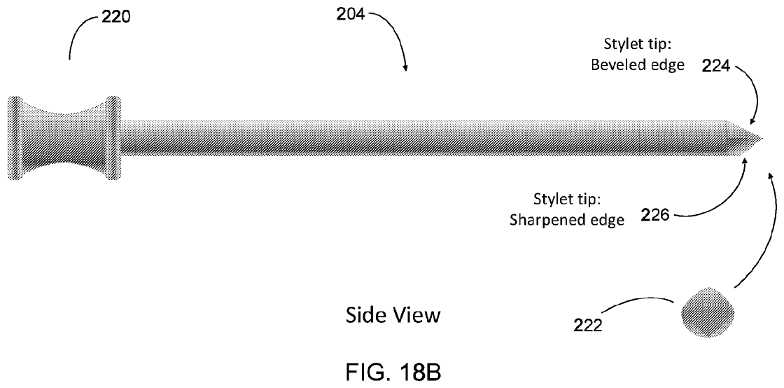

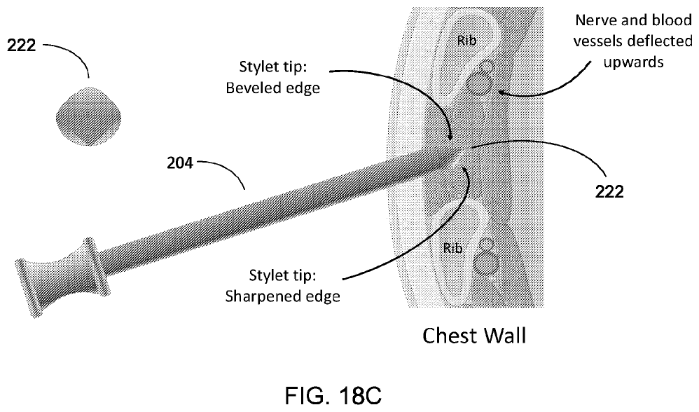

FIGS. 18A and B illustrate the distal stylet tip's 222 combined wedge design employing exemplary views of the beveled edge 224 and sharpened edge 226. This provides a point to easily cut through the skin during deployment of the chest tube device 200. While not shown, it is contemplated that the stylet distal tip 222 can comprise other shapes or points to better cut through the skin when slidably deployed through the chest tube 202. The combination of distal tip design incorporating, for example both the beveled edge 224 and sharpened edge 226 obviates the need for additional instruments and work-effort, respectively, to place chest tube device 200. Therefore, placing the chest tube device 200 is both simplified and expedited owing to the multi-faceted design of the distal stylet tip 222.

FIG. 18C illustrates the multiple exemplary functions of the distal stylet tip 222 in accordance with one or more embodiments. The chest tube device 200 is pushed against the skin of the chest wall where it meets resistance. The sharpened edge 226 of the distal stylet tip 222 cuts through the tissue facilitating entry. The upper beveled edge 224 deflects vital tissue up and away from the distal stylet tip 222 as a measure of safety. The underlying sharpened edge 226 of the distal stylet tip 222 simultaneously cuts through the fibrous fascial protective layer of the rib muscles (i.e., intercostal muscles) and the thick, resistive fibrous inner chest wall layer of tissue (i.e., the parietal pleura) protecting pleural space the lung within.

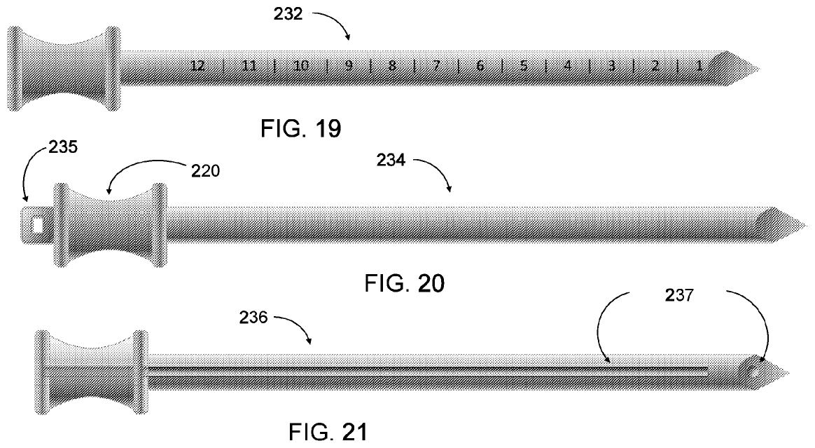

In the embodiment shown in FIG. 19 illustrates an alternate stylet 232 with markings (i.e., gradations) along the stylet shaft 221 for measuring distances from the stylet tip. Additionally, the markings may be luminescent for use in low-light conditions. Thus, the chest tube 202 can be opaque or clear allowing the user to read measurements from the ruler while deploying the stylet 240. This allows measurements to be made using just the stylet as a measuring device. Distances from the tip when in the tissue can add a margin of safety and expediency. Similarly, lengths of any item can be measured in-hand without the need for finding or using another measuring device especially under times of duress.

FIG. 20 illustrates a further alternate stylet 234 in accordance with one or more embodiments. The stylet 234 has a grommet 235 in the stylet handle 220. This grommet allows the stylet to be recycled and further used, for example, to attach and/or secure various items. This also allows fastening the stylet to a person or gear to prevent loss. The shape of the grommet may also be fashioned for use as a tool such as in opening or turning any respectively designed equipment in this or any other intended embodiment.

In the embodiment shown in FIG. 21, the stylet 236 has a hollow lumen 237 running along its entire length. This facilitates rapid egress and decompression when deployed for trapped fluid. This immediate audible rush of air, for example, alerts the user that the pleural space has been entered, the pressure temporarily relieved and the heart allowed to fill adequately. Additionally, the lumen allows the chest tube device 200 to be deployed using a guidewire as a measure of added safety when the anatomy and/or habitus make initial placement questionable.

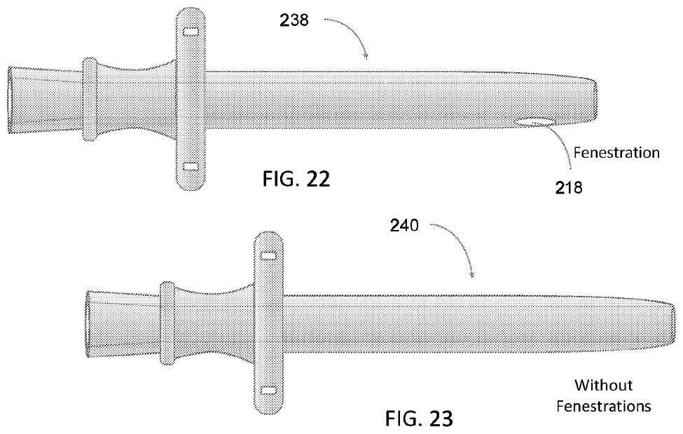

FIG. 22 has a single fenestration slightly proximal to the distal tapered chest tube tip 208. This functions to add a second egress point for fluids. This can be helpful when the chest tube tip is clogged by inspissated bodily fluids or blocked by tissue. But, with only one fenestration it mitigates the risk of losing of suction if there were more holes in the tube.

FIG. 23 has no fenestrations that can impede passage of the chest tube 202 through tissue. As the chest tube portion 202 is inserted the natural integrity and recoil of the surrounding tissue may be caught and shorn. Without the added fenestration, the chest tube is less likely to traumatize the surrounding tissue or get clogged. In a similar manner, the distal chest tube tip 208 is tapered towards the distal end making an angle in line with the angle created by the edges of the distal stylet tip 222. This allows the cross-sectional area of the chest tube tip 208 to be slightly smaller and in continuity with the stylet further decreasing the work-effort to deploy the chest tube device 200 and further reduce damage to the skin and tissue upon initial placement.

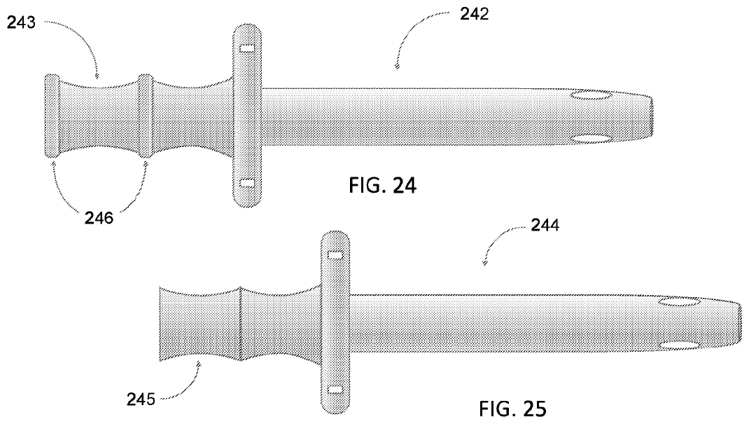

FIGS. 24 and 25 show further alternative exemplary proximal ends of the chest tube device 202. These figures illustrate the addition of a second contoured finger depression over the flared proximal chest tube lumen 206 for added comfort and grip. The chest tube embodiment 242 illustrated in FIG. 24 includes both the additional contoured depression 243 and includes the tactile ridge 246 for added measure in mitigating slippage of the device. FIG. 25 shows the chest tube embodiment 244 with the additional contoured depression 245 but lacking the tactile ridges which may confer less discomfort for different hand/finger sizes.

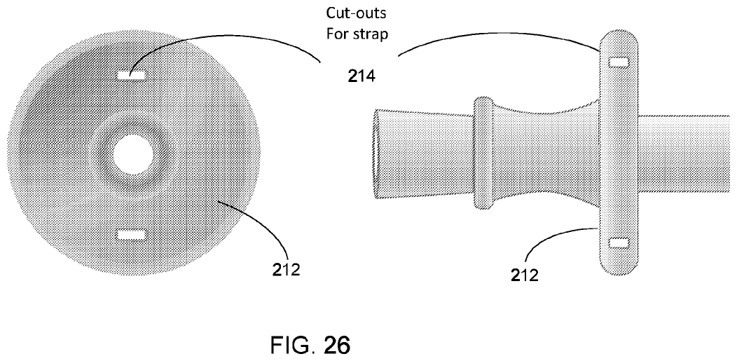

FIG. 26 illustrates the exemplary chest tube flange 212 of the chest tube device 200. The chest tube flange prevents the chest tube device 200 from being placed too far inside the chest. The chest tube flange 212 confers multiple benefits to the chest tube device 200 by: 1) preventing over-penetration (over-insertion) and distal damage to critical organs towards the center of the chest (e.g., the heart and great vessels), 2) allowing placement under poor lighting conditions by rigidly fixing an appropriate length of the chest tube device 200 needed for efficacious placement without excess, 3) placing the chest tube device 200 rapidly under stressful, life-threatening conditions given the tactile feedback the natural block of the flange coupled with the intuitive action of pushing a rigid, straight implement (e.g., a rod, skewer, spike or knife) through material (i.e., tissue), 4) providing a large, simplified anchoring point for quickly and effectively securing the chest tube portion to the patient (e.g., with the included strap placed around the chest or, if necessary, with sutures placed through the skin and flange) at the skin level to prevent inadvertent withdrawal and loss of the deployed chest tube, and 5) obviating the need for extensive formal training and continual, ongoing practice given the simplified nature of rapidly deploying and securing the chest tube device 202 without additional equipment or instruments needed in the current state of the art for tube thoracostomy. In addition, the shape of the flange and the support additional material of the contoured finger depression 230 confers to the strength and rigidity to the flange in preventing eversion of the flange edges, material degradation and risk of breaking during packaging, travel and eventual placement.

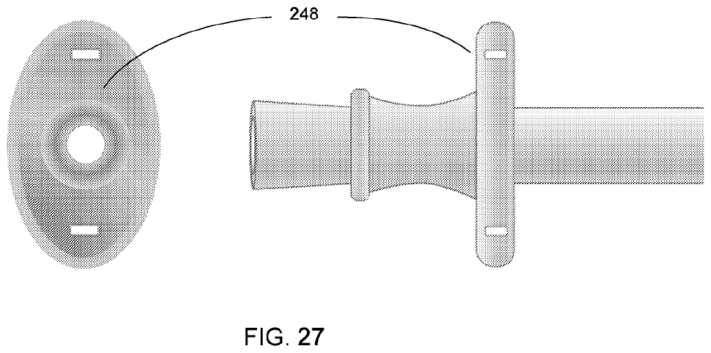

FIG. 27 illustrates an exemplary embodiment of chest tube flange 248. The oval shape allows less material to be used in a slimmer device profile. The chest tube can be deployed with the long axis of the ovular flange along a line from head to toe to reduce irritation to the patient, while still providing adequate material and strength for securing the chest tube device to the patient.

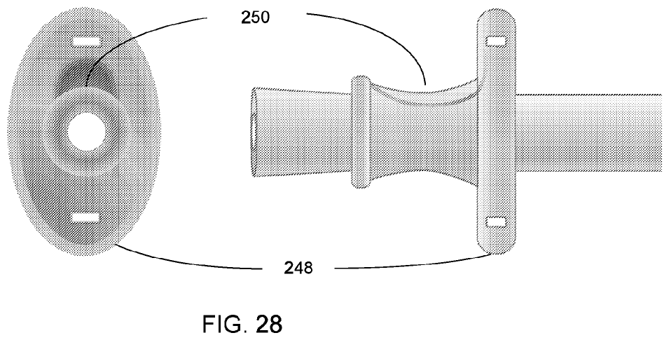

FIG. 28 show another exemplary embodiment of the oval shaped flange 248 coupled with a finger groove 250 for resting the thumb. This provides additional strength and support for insertion against resistance when deployed using the thumb. As illustrated in the figure, the thumb groove lies parallel to and is centered along the long axis of the oval flange 248 at one end. In this embodiment, the thumb groove is fashioned as a recessed surface groove 250 in both the ovular flange 248 and the contoured finger depression 230 of the chest tube. In this embodiment, directionality is afforded to the chest tube device 200 that greatly aids in the safety and efficacy of the device. With the directionality provided by the combination of the oval shaped flange 248 and thumb groove 250, the flat surface of the beveled edge 224 of the stylet tip can be effectively and safely directed through the chest wall and into the lung space whereby greatly reducing the risk of damage to vital structures (e.g., the neurovascular bundle on the underside of the ribs) and critical organs. Moreover, this contributes a further decrease in work-effort and risk of awkward or dangerous placement by providing another surface on the chest tube device for control of the chest tube device 200 during placement.

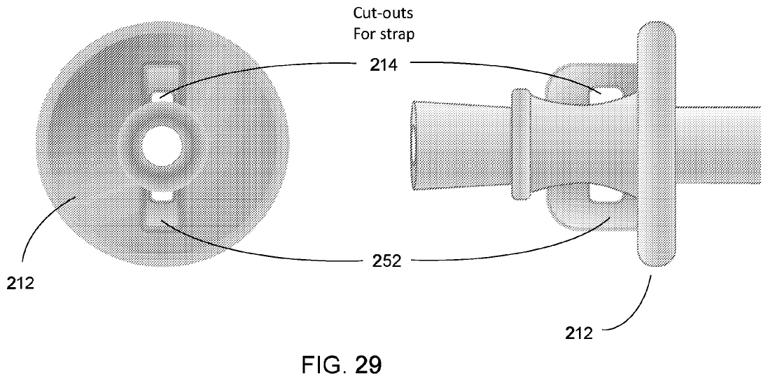

FIG. 29 illustrates another exemplary embodiment of the chest tube device utilizing separate attachment points (i.e., grommets) 252 incorporating both the flange 212 and finger groove 230 of the chest tube. This embodiment illustrates variable placement of the attachment cut-outs whereby the strap is no longer in contact with the skin. This decreases skin irritation and breakdown caused by the strap rubbing against the skin. This also decreases the risk of skin infection. In the exemplary embodiment shown, the grommets 252 extend distally from the flange 212, and radially outward from the outer surface of the chest tube.

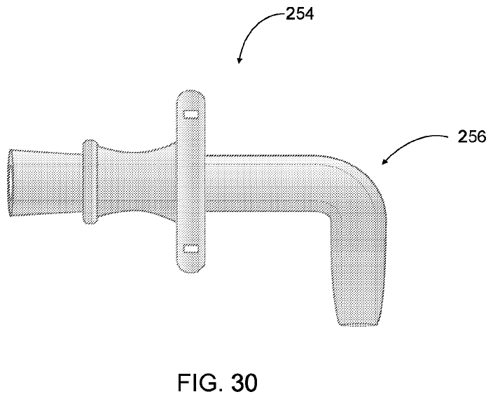

In the FIG. 30 embodiment, an alternative exemplary right-angle tube 254 in accordance with one or more embodiments. The chest tube 254 includes a distal end 256 curved 90′ to allow directional orientation, for example, to be used when liquid within the pleural cavity is not easily accessed by a “straight” chest tube 202 due anatomical location. The right-angle tube 254 can comprise a soft material, e.g., a soft silastic plastic (a flexible, inert silicone elastomer) that has been molded into shape. A radio-opaque strip 215 along “back” or “spine” of tube identifies tube curvature. This strip 215 can include a ruler or other measurement system to indicate to the user the insertion depth of the chest tube 254. Additionally, the chest tube 254 has a flange 212 proximal to the right angle bend in the chest tube 254. While the embodiment in FIG. 30 does not comprise any fenestration along the distal end oright-angle tube 254, alternative embodiments may include fenestrations 218 as shown in previous figures.

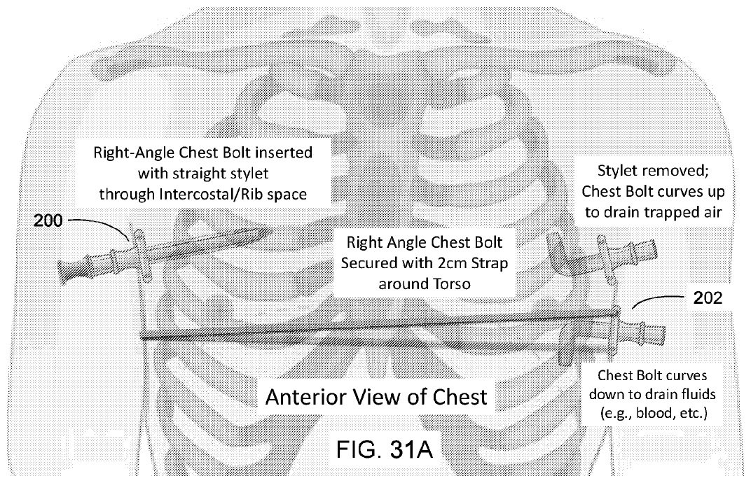

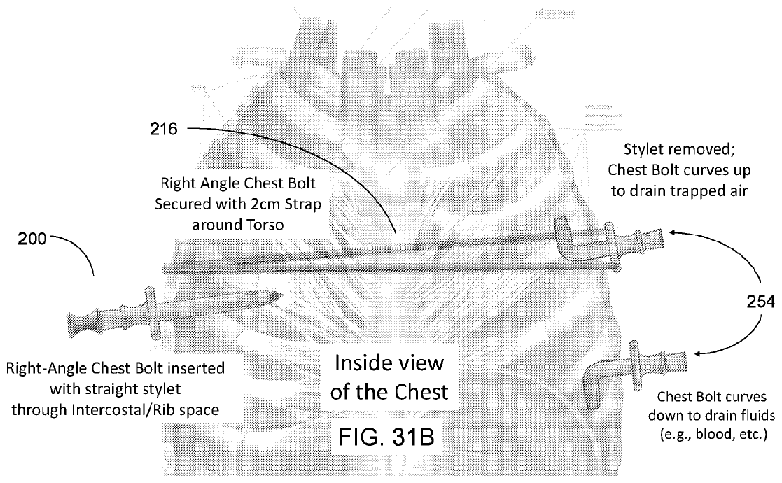

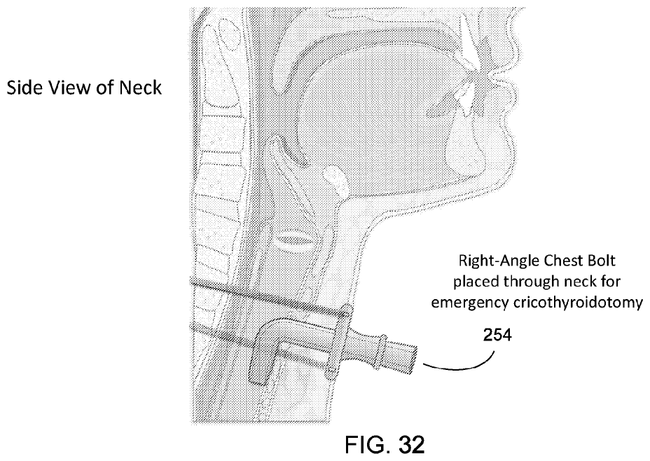

In some embodiments, the tube 254 comprises a bend of less, or more, than 90′. The (straight) stylet 204 can be employed, without a tube 254, to pierce the patient and initiate a pathway for the tube 254 insertion; thereafter the tube 254 can be partially inserted, e.g., up to the bend location, and articulated by the physician to change orientation, and further insertion angle of the tube within the patient such that the tube results in the positioning shown in FIGS. 31A and 31B, similar to the embodiments as shown in FIGS. 15A and 15B where the exemplary chest tube device 200 and chest tube 254 are inserted through the intercostal rib space to drain fluid from within the pleural cavity.

Additionally or alternatively, in some embodiments the tube 254 can be originally configured as a straight tube, but upon insertion within the patient, the tube “converts” or articulates to the bent configuration 254, as shown in FIGS. 31A & 31B. For example, the tube can be formed from shape-memory material such that when inserted into the body, the tube transitions from a martensitic (straight) configuration to an austenitic (bent) configuration. As shown in FIGS. 31A & 31B, the tube 254 can then be positioned cephalad (i.e., towards the head or “up”), which allows pointing towards trapped air within the superior portion of the lung space. The tube 254 can also be positioned caudally (i.e., towards the feet or “down”), which allows pointing the tube, for example, towards trapped fluid at the base of the lung space. The right-angle tube 254 is secured by strap 216 placed around the patient's torso to ensure stability of the tube 254 during deployment.

In some applications, the (straight) stylet is inserted into an initially-biased bent chest tube as shown in FIGS. 10 and 30, which temporarily straightens the tube to mimic the shape of the stylet. The assembly is inserted into a desired location (and depth) of the patient, and as the stylet is withdrawn, the chest tube resumes its initial/biased configuration to return to a bent (non-linear) shape. Additionally or alternatively, the tube can be inserted as a generally linear tube, and the stylet removed while the tube maintains a linear/straight configuration. Thereafter, an operator can manipulate the tube to impart a desired shape/contour for the particular patient geometry. For example, the tube can include a plurality of wires or tension mechanisms that extend from the distal end (and/or flange), which allow an operator to selectively, and independently, alter (e.g. shorten) the length of the wire-thereby causing the tube to bend/articulate in a predetermined direction. That is, the operator can pull a wire on the lower/bottom side of the tube shown in FIG. 30, which will cause the tube to bend/articulate into the configuration shown.

As shown in FIG. 32, the right-angle tube 254 can also be used for rapid field tracheostomy (i.e., breathing tube through anterior neck into trachea). The tube 254 thus provides an “all-in-one” field system of management when combined with a rigid, inert stylet for emergency airway and breathing issues in a multitude of clinical scenarios.

Having thus described several illustrative embodiments, it is to be appreciated that various alterations, modifications, and improvements will readily occur to those skilled in the art. Such alterations, modifications, and improvements are intended to form a part of this disclosure and are intended to be within the spirit and scope of this disclosure. While some examples presented herein involve specific combinations of functions or structural elements, it should be understood that those functions and elements may be combined in other ways according to the present disclosure to accomplish the same or different objectives. In particular, acts, elements, and features discussed in connection with one embodiment are not intended to be excluded from similar or other roles in other embodiments. Additionally, elements and components described herein may be further divided into additional components or joined together to form fewer components for performing the same functions.

Accordingly, the foregoing description and attached drawings are by way of example only, and are not intended to be limiting.

What is claimed is:

1. A chest tube device, comprising:
a chest tube having a proximal end and a distal end, and a lumen extending therethrough, the proximal end having an opening with a diameter larger than a diameter of the lumen and the distal end having a tapered tip;
a circular flange having an inner diameter equal to an outer diameter of the chest tube, the flange positioned on the chest tube to limit insertion of the chest tube during deployment of the chest tube device, the circular flange having at least one cutout configured to receive a strap to fix the chest tube device to a patient; and
a stylet having a proximal end and a distal end, the proximal end having a handle for a user to grip during deployment, the distal end having a beveled tip, the stylet further having a diameter smaller than a diameter of the lumen of the chest tube;
wherein the stylet is slidably movable through the chest tube such that the distal end of the stylet protrudes from the distal end of the chest tube, and the handle of the stylet is proximal to the proximal end of the chest tube.

2. A chest tube device, comprising:
a chest tube having a proximal end and a distal end, and a lumen extending therethrough, the proximal end having an opening with a diameter larger than a diameter of the lumen and the distal end having a tapered tip, the chest tube including a plurality of fenestrations disposed about the distal end of the tube;
a flange disposed on an outer surface of the chest tube and proximal of the plurality of fenestrations, the flange including at least one sidewall extending perpendicularly outward from the chest tube to limit insertion of the chest tube during deployment of the chest tube device, the flange having at least one cutout configured to receive a strap to fix the chest tube device to a patient; and
a removable stylet having a proximal end and a distal end, the proximal end having a handle, the distal end having a beveled tip, the stylet further having a diameter smaller than a diameter of the lumen of the chest tube;
wherein the stylet is slidably movable through the chest tube such that the distal end of the stylet protrudes from the distal end of the chest tube, and the handle of the stylet is proximal to the proximal end of the chest tube.

3. A chest tube device, comprising:
a chest tube having a lumen extending therethrough from a proximal end of the chest tube to a distal end of the chest tube, said chest tube including a flange between the proximal end and the distal end to limit insertion of the chest tube into a patient's body during deployment; and
a stylet having a handle and a shaft, said shaft having a proximal end connected to the handle and a free opposite distal end having a beveled tip, wherein the shaft of the stylet is slidably positioned in the lumen of the chest tube with the beveled tip protruding from the distal end of the chest tube to facilitate insertion of the chest tube into the patient's body, wherein the stylet is removable from the chest tube after deployment.

4. The device of claim 1, wherein the proximal end of the chest tube is flared to facilitate insertion of an adapter for suction device.

5. The device of claim 1, wherein the flange is located 8 to 10 cm from the distal end of the chest tube.

6. The device of claim 1, wherein the flange includes cutouts for attachment of a strap to secure the chest tube to the patient's body.

7. The device of claim 1, wherein the chest tube includes one or more fenestrations between the flange and the distal end of the chest tube for egress of fluids.

8. The device of claim 1, the one or more fenestrations include a proximal hole 6 cm away from the flange towards the distal end of the chest tube.

9. The device of claim 1, wherein the chest tube comprises a flexible inert silicone elastomer material.

10. The device of claim 1, wherein the stylet comprises a high density polyethylene material.

11. The device of claim 1, wherein the handle of the stylet has a raised undulating profile.

12. The device of claim 1, wherein the handle of the stylet is knurled with finger grooves.

13. The device of claim 1, where in the end of the handle proximate the shaft is flat and curved in word at the edges thereof to engage in accommodate the proximal end of the chest tube.

14. The device of claim 1, wherein the tip of the stylet has a triangular shape.

15. The device of claim 1, wherein the handle of the stylet includes an eyelet for attaching strap or cord.

16. The device of claim 1, wherein the handle of the stylet includes a flat end to facilitate hammering.

17. The device of claim 1, wherein the shaft of the stylet includes ruler markings.

18. The device of claim 1, wherein the markings are phosphorescent for use in low-light conditions.

19. The device of claim 1, wherein the chest tube has a right angle shape.

20. The device of claim 1, wherein the chest tube includes a radio-opaque marker to identify tube curvature.
